(19) United States
(12) Patent Application Publication
(10) Pub. No.: US 2004/0258628 A1
(43) Pub. Date: Dec. 23, 2004

(54) Self-foaming, foam-type, post-foaming or foamable cosmetic or dermatological preparations containing siloxane elastomers

(75) Inventors: Heidi Riedel, Hamburg (DE); Rainer Kropke, Schenefeld (DE); Andreas Bleckmann, Ahrensburg (DE); Ghita Lanzendorfer, Hamburg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/846,912

(22) Filed: May 14, 2004

Related U.S. Application Data
(63) Continuation of application No. PCT/EP02/10453, filed on Sep. 18, 2002.

(30) Foreign Application Priority Data
Nov. 14, 2001 (DE) 101 55 792.2

(51) Int. Cl. A61K 7/00

(52) U.S. Cl. 424/47

(57) ABSTRACT

The present invention is a self-foaming, foam-type, post-foaming or foamable cosmetic or dermatological preparation comprising at least one siloxane elastomer selected from the group of siloxane elastomers obtainable by reacting vinyl-terminated polymethylsiloxane with methylhydro-dimethylsiloxane or by reacting hydroxy-terminated dimethylpolysiloxane with trimethylsiloxy-terminated methylpolysiloxane. The present invention also includes a method of preparing foam comprising combining the cosmetic or dermatological preparation with at least one gas or propellant by releasing the preparation and the gas or propellant from a pressurized gas container, and a method for increasing the stability of a foam comprising adding at least one siloxane elastomer.

### CROSS-REFERENCE TO RELATED APPLICATIONS

This is a continuation application of PCT/EP02/10453, filed Sep. 18, 2002, which is incorporated herein by reference in its entirety, and also claims the benefit of German Priority Application No.101 55 792.2, filed Nov. 14, 2001.

### FIELD OF THE INVENTION

The present invention relates to self-foaming, foam-type, post-foaming or foamable cosmetic and dermatological preparations, in particular skincare cosmetic and dermatological preparations.

### BACKGROUND OF THE INVENTION

Foams or foam-type preparations are a type of disperse system.

By far the most important and best known disperse system are emulsions. Emulsions are two- or multi-phase systems of two or more liquids which are insoluble or only slightly soluble in one another. The liquids (pure or as solutions) are present in an emulsion in a more or less fine distribution, which generally has only limited stability.

Foams are structures of gas-filled, spherical or polyhedral cells which are delimited by liquid, semi-liquid, high-viscosity or solid cell ribs. The cell ribs, connected via points of intersection, form a continuous framework. The foam lamellae stretch between the cell ribs (closed-cell foam). If the foam lamellae are disturbed or if they flow back into the cell rib at the end of foam formation, an open-cell foam is obtained. Foams are also thermodynamically unstable since a reduction in the surface area leads to the production of surface energy. The stability and thus the existence of a foam is thus dependent on to what extent it is possible to prevent its self-destruction.

Cosmetic foams are usually dispersed systems of liquids and gases, where the liquid represents the dispersant and the gas represents the dispersed substance. Foams of low-viscosity liquids are temporarily stabilized by surface-active substances (surfactants, foam stabilizers). Because of their large internal surface area, such surfactant foams have a high adsorption capacity, which is utilized, for example, in cleaning and washing operations. Accordingly, cosmetic foams are used, in particular, in the fields of cleansing, for example as shaving foam, and of hair care.

To generate foam, gas is bubbled into suitable liquids, or foam formation is achieved by vigorously beating, shaking, spraying or stirring the liquid in the gas atmosphere in question, provided that the liquids comprise suitable surfactants or other interface-active substances (“foam formers”), which, apart from interfacial activity, also have a certain film-forming ability.

Cosmetic foams have the advantage over other cosmetic preparations of permitting a fine distribution of active ingredients on the skin. However, cosmetic foams can generally only be achieved using particular surfactants, which, moreover, are often not well tolerated by the skin.

A disadvantage of the prior art is that such foams have only low stability, for which reason they usually collapse within approximately 24 hours. A requirement of cosmetic preparations, however, is that they have stability for years, as far as possible. This problem is generally taken into account by the fact that the consumer produces the actual foam himself just before use using a suitable spray system, for which purpose, for example, it is possible to use spray cans in which a liquefied pressurized gas serves as propellant gas. Upon opening the pressure valve, the propellant liquid mixture escapes through a fine nozzle, and the propellant evaporates, leaving behind a foam.

Post-foaming cosmetic preparations are also known per se. They are firstly applied to the skin from an aerosol container in flowable form and, after a short delay, develop the actual foam only once they are on the skin under the effect of the post-foaming agent present, for example a shaving foam. Post-foaming preparations are often in specific formulation forms, such as, for example, post-foaming shaving gels or the like.

However, the prior art does not include any sort of cosmetic or dermatological preparations which could be foamed as early as during the preparation and nevertheless have a sufficiently high stability in order to be packaged in the usual manner, stored and put onto the market.

### SUMMARY OF THE INVENTION

An object of the present invention was therefore to enrich the prior art and to provide cosmetic or dermatological self-foaming or foam-type preparations which do not have the disadvantages of the prior art.

German laid-open specification DE 197 54 659 discloses that carbon dioxide is a suitable active ingredient for stabilizing or increasing the epidermal ceramide synthesis rate, which may serve to enhance the permeability barrier, reduce the transepidermal water loss and increase the relative skin moisture. To treat the skin, the CO₂ is, for example, dissolved in water, which is then used to rinse the skin. However, the prior art hitherto does not include any sort of cosmetic or dermatological bases in which a gaseous active ingredient could be incorporated in an adequate, i.e. effective, concentration.

It was thus a further object of the present invention to find cosmetic or dermatological bases into which effective amounts of gaseous active ingredients can be incorporated.

The use of solid elastomeric polyorganosiloxanes or organopolysiloxanes, referred to below as siloxane elastomers, in cosmetic preparations is known per se and has gained importance in recent years. Besides being used in cosmetics, these substances have been used in foods and animal feeds, medicaments, impregnating compositions, lubricants and so on. Siloxane elastomers are partially or completely crosslinked and in most cases have a three-dimensional structure. They are obtainable by reacting vinyl-terminal polymethylsiloxane and methylhydrodimethylsiloxane

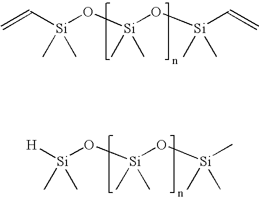

or else by reacting hydroxy-terminal dimethylpolysiloxane and trimethylsiloxy-terminal methylpolysiloxane:

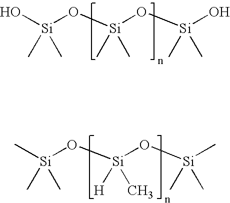

These siloxane elastomers are used, for example, for adjusting the rheological properties of a preparation. Siloxane elastomers of this type are described, for example, in European patent specification 295 886 and U.S. Pat. No. 5,266,321, which disclose the use of these substances in face-cleansing compositions and oil-containing makeup products. The nature of the siloxane elastomers is also described in more detail in the specification. The use of the siloxane elastomers in cosmetic preparations is in particular due to their pleasant sensory properties, the resulting products are described as velvety, powdery or matting. In addition, they have stabilizing effects on formulations with high oil contents and low water contents of at most 5% by weight. When formulating the abovementioned products, the problem often arises that the siloxane elastomers are incompatible with other frequently used components, which leads to unsatisfactory long-term stability of the products.

A further object of the present invention was therefore to provide cosmetic or dermatological self-foaming or foam-type preparations which have good long-term storage stabilities and pleasant sensory properties.

It was surprising and could not have been foreseen by the person skilled in the art that self-foaming, foam-type, post-foaming or foamable cosmetic or dermatological preparations comprising at least one siloxane elastomer chosen from the group of siloxane elastomers obtainable by reacting vinyl-terminal polymethylsiloxane and methylhydrodimethylsiloxane or by reacting hydroxy-terminal dimethylpolysiloxane and trimethylsiloxy-terminal methylpolysiloxane overcome the disadvantages of the prior art.

### DETAILED DESCRIPTION OF THE PREFERRED EMBODIMENTS

For the purposes of the present invention, “self-foaming”, “foam-type”, “post-foaming” and “foamable” are understood as meaning preparations from which foams can in principle be prepared—whether during the preparation process, whether during use by the consumer or in another way—by introducing one or more gases. In such foams, the gas bubbles are present in (any) distributed form in one (or more) liquid phase(s), where the (foamed) preparations do not necessarily have to have the appearance of a foam in macroscopic terms. (Foamed) cosmetic or dermatological preparations according to the invention (for the sake of simplicity also referred to below as foams) may, for example, be macroscopically visibly dispersed systems of gases dispersed in liquids. The foam character can, however, for example, be visible only under a (light) microscope. Moreover, foams according to the invention are—particularly when the gas bubbles are too small to be recognized under a light microscope—also recognizable from the sharp increase in volume of the system.

According to the prior art, self-foaming, foam-type, post-foaming or foamable cosmetic emulsions could not be formulated or prepared industrially without the use of particular surfactants. This was true particularly for systems based on classic emulsifiers. According to the prior art, systems of this type, with the addition of propellant gas, developed exclusively aqueous-moist foams, which rapidly broke following application.

As a result of the invention, a rich, compact cream foam is obtainable for the first time which is characterized by a long storage period and by an extraordinarily high stability and a compact appearance.

As a result of the use of the siloxane elastomers according to the invention, the introduction of gases is aided, and a stabilizing and significantly foam-boosting effect is achieved over a prolonged storage period, even at relatively high temperatures (e.g. 40° C.). It was particularly surprising in this regard that the use of particular surfactants can be dispensed with. The introduction of gases is, surprisingly, extraordinarily increased compared with the prior art. Thus, for example, foam boosting with up to 100% increased gas volume can be achieved without using foaming agents customary according to the prior art, such as surfactants.

As a result of this, it is possible for the first time to stably generate formulations with an excellent, novel type of cosmetic activity and with an extraordinarily high gas volume (air or other gases, such as oxygen, carbon dioxide, nitrogen, helium, argon, etc.) over a long storage period at high temperatures. At the same time, the preparations according to the invention are characterized by above-average skincare and very good sensory properties.

The present invention thus further provides for the use of one or more siloxane elastomers chosen from the group of siloxane elastomers obtainable by reacting vinyl-terminal polymethylsiloxane and methylhydrodimethylsiloxane or by reacting hydroxy-terminal dimethylpolysiloxane and trimethylsiloxy-terminal methylpolysiloxane for boosting the foam of self-foaming, foam-type, post-foaming or foamable cosmetic and dermatological preparations.

For the purposes of the present invention, “foam boosting” is understood as meaning that the introduction of gases into the foams according to the invention is extraordinarily increased compared to the introduction into otherwise identical preparations which do not comprise siloxane elastomers according to the invention. The foams according to the invention can, accordingly, take up a significantly greater gas volume than preparations which do not comprise siloxane elastomers according to the invention.

Moreover, “foam boosting” means that the stability of the foamed preparations (the “foam stability”) compared with otherwise identical preparations which do not comprise siloxane elastomers according to the invention is significantly improved, i.e. as a result of the use according to the invention, breaking of the foams is delayed.

In addition, for the purposes of the present invention, “foam boosting” is understood as meaning that the cosmetic properties of the foams according to the invention compared with preparations which do not comprise siloxane elastomers according to the invention are also significantly improved: thus, the use according to the invention gives rich, solid foams (“foam creams”) which, despite their compactness and richness, can be spread easily and absorb rapidly.

The preparations according to the invention are entirely satisfactory preparations in every respect. It was particularly surprising that the foam-type preparations according to the invention are extraordinarily stable, even in cases of an unusually high gas volume. Accordingly, they are particularly suitable for use as bases for preparation forms having diverse intended uses. The preparations according to the invention have very good sensory properties, such as, for example, extensibility on the skin or the ability to be absorbed into the skin and, moreover, are characterized by above-average skincare.

It is preferred when the siloxane elastomers according to the invention are chosen from one of the following groups of siloxane elastomers:

(a) siloxane elastomers which contain the units R₂SiO and RSiO_{1.5} and/or R₃SiO_{0.5} and/or SiO₂,

where the individual radicals R are in each case independently of one another hydrogen, C₁₋₂₄-alkyl (such as, for example, methyl, ethyl, propyl) or aryl (such as, for example phenyl or tolyl), alkenyl (such as, for example, vinyl), and the weight ratio of the units R₂SiO to RSiO_{1.5} is chosen from the range from 1:1 to 30:1;

(b) siloxane elastomers which are insoluble and swellable in silicone oil and are obtainable by the addition reaction of an organopolysiloxane (1) which contains silicon-bonded hydrogen with an organopolysiloxane (2) which contains unsaturated aliphatic groups,

where the quantitative fractions used are chosen such that the amount of hydrogen in the organopolysiloxane (1) or of unsaturated aliphatic groups in the organopolysiloxane (2)

is in the range from 1 to 20 mol % when the organopolysiloxane is non-cyclic and

is in the range from 1 to 50 mol % when the organopolysiloxane is cyclic.

The siloxane elastomers according to the invention are advantageously present in the form of spherical powders or in the form of gels.

Advantageous siloxane elastomers according to the invention present in the form of spherical powders are the crosspolymers with the INCI name DimethiconeNinyl Dimethicone, for example that obtainable from DOW CORNING under the trade name DOW CORNING 9506 Powder.

It is particularly preferred when the organopolysiloxane elastomer is used in combination with oils from hydrocarbons of animal and vegetable origin, synthetic oils, synthetic esters, synthetic ethers or mixtures thereof.

It is very particularly preferred when the organopolysiloxane elastomer is used in combination with unbranched silicon oils which are pasty or liquid at room temperature, or cyclic silicone oils or mixtures thereof. Organopolysiloxane elastomers with the INCI name Dimethicone/Polysilicone-11, very particularly the Gransil grades GCM, GCM-5, DMG-6, CSE Gel, PM-Gel, LTX, ININ Gel, AM-18 Gel or DMCM-5 available from Grant Industries Inc. are particularly advantageous.

It is very extraordinarily preferred when the organopolysiloxane elastomer is used in the form of a gel of organopolysiloxane elastomer and a lipid phase, where the content of the organopolysiloxane elastomer in the gel is 1 to 80% by weight, preferably 0.1 to 60% by weight, in each case based on the total weight of the gel.

It is advantageous for the purposes of the present invention to choose the total amount of siloxane elastomers (active content) from the range from 0.01 to 10% by weight, advantageously from 0.1 to 5% by weight, in each case based on the total weight of the formulation.

Particularly advantageous preparations for the purposes of the present invention comprise an emulsifier system which consists of

A. at least one emulsifier A chosen from the group of completely, partially or unneutralized, branched or unbranched, saturated or unsaturated fatty acids with a chain length of from 10 to 40 carbon atoms,

B. at least one emulsifier B chosen from the group of polyethoxylated fatty acid esters with a chain length of from 10 to 40 carbon atoms and with a degree of ethoxylation of from 5 to 100 and

C. at least one coemulsifier C chosen from the group of saturated or unsaturated, branched or unbranched fatty alcohols with a chain length of from 10 to 40 carbon atoms.

The emulsifier A is preferably chosen from the group of fatty acids which have been completely or partially neutralized with customary alkalis (such as, for example, sodium hydroxide, potassium hydroxide, sodium carbonate, potassium carbonate, and mono- and triethanolamine). Stearic acid and stearates, isostearic acid and isostearates, palmitic acid and palmitates, and myristic acid and myristates, for example, are particularly advantageous.

The emulsifier B is preferably chosen from the following group: PEG-9 stearate, PEG-8 distearate, PEG-20 stearate, PEG-8 stearate, PEG-8 oleate, PEG-25 glyceryl trioleate, PEG-40 sorbitan lanolate, PEG-15 glyceryl ricinoleate, PEG-20 glyceryl stearate, PEG-20 glyceryl isostearate, PEG-20 glyceryl oleate, PEG-20 stearate, PEG-20 methylglucose sesquistearate, PEG-30 glyceryl isostearate, PEG-20 glyceryl laurate, PEG-30 stearate, PEG-30 glyceryl stearate, PEG-40 stearate, PEG-30 glyceryl laurate, PEG-50 stearate, PEG-100 stearate, PEG-150 laurate. Particularly advantageous are, for example, polylethoxylated stearic esters.

The coemulsifier C is preferably chosen according to the invention from the following group: behenyl alcohol (C₂₂H₄₅OH), cetearyl alcohol [a mixture of cetyl alcohol (C₁₆H₃₃OH) and stearyl alcohol (C₁₈H₃₇OH)], lanolin alcohols (wool wax alcohols, which are the unsaponifiable alcohol fraction of wool wax which is obtained following the saponification of wool wax). Particular preference is given to cetyl alcohol and cetylstearyl alcohol.

It is advantageous according to the invention to choose the weight ratios of emulsifier A to emulsifier B to coemulsifier C (A:B:C) as a:b:c, where a, b and c, independently of one another, may be rational numbers from 1 to 5, preferably from 1 to 3. Particular preference is given to a weight ratio of approximately 1:1:1.

It is advantageous for the purposes of the present invention to choose the total amount of emulsifiers A and B and of coemulsifier C from the range from 2 to 20% by weight, advantageously from 5 to 15% by weight, in particular from 7 to 13% by weight, in each case based on the total weight of the formulation.

For the purposes of the present invention, it is particularly preferred if the gas phase of the preparations comprises carbon dioxide or consists entirely of carbon dioxide. It is particularly advantageous if carbon dioxide is a or the active ingredient in the preparations according to the invention.

Compositions according to the invention develop, even during their preparation—for example during stirring or upon homogenization—into fine-bubble foams. According to the invention, fine-bubble, rich foams of excellent cosmetic elegance are obtainable. Furthermore, preparations which are particularly well tolerated by the skin are obtainable according to the invention, where valuable ingredients can be distributed on the skin in a particularly good manner.

It may be advantageous, although it is not necessary, for the formulations according to the present invention to comprise further emulsifiers. Preference is given to using those emulsifiers which are suitable for the preparation of W/O emulsions, it being possible for these to be present either individually or else in any combinations with one another.

Preferably, for the purposes of the present invention, the further emulsifier(s) is/are chosen from the group of hydrophilic emulsifiers. According to the invention, particular preference is given to mono-, di- and tri-fatty acid esters of sorbitol.

The total amount of further emulsifiers is, according to the invention, advantageously chosen to be less than 5% by weight, based on the total weight of the formulation.

The list of given further emulsifiers which can be used for the purposes of the present invention is not of course intended to be limiting.

Particularly advantageous preparations for the purposes of the present invention are free from mono- or diglyceryl fatty acid esters. Particular preference is given to preparations according to the invention which comprise no glyceryl stearate, glyceryl isostearate, glyceryl diisostearate, glyceryl oleate, glyceryl palmitate, glyceryl myristate, glyceryl lanolate and glyceryl laurate.

The oil phase of the preparations according to the invention is advantageously chosen from the group of nonpolar lipids having a polarity of >30 mN/m and of the cyclic or linear silicone oils. Particularly advantageous nonpolar lipids for the purposes of the present invention are those listed below.

| | | | Polarity |
|---|---|---|---|
| Manufacturer | Trade name | INCI name | mN/m |
| Total SA | Ecolane 130 | Cycloparaffin | 49.1 |
| Neste PAO N.V. | Nexbase 2006 FG | Polydecene | 46.7 |
| (Supplier Hansen & Rosenthal) | | | |
| Chemische Fabrik Lehrte | Polysynlane | Hydrogenated | 44.7 |
| | | Polyisobutene | |
| Wacker | Wacker Silicone | Polydimethylsiloxane | 46.5 |
| | oil AK 50 | | |
| EC Erdölchemie (Supplier | Solvent ICH | Isohexadecane | 43.8 |
| Bayer AG) | | | |
| DEA Mineral oil (Supplier | Pionier 2076 | Mineral Oil | 43.7 |
| Hansen & Rosenthal) | | | |
| Tudapetrol | | | |
| DEA Mineral oil (Supplier | Pionier 6301 | Mineral Oil | 43.7 |
| Hansen & Rosenthal) | | | |
| Tudapetrol | | | |
| Wacker | Wacker Silicone | Polydimethylsiloxane | 42.4 |
| | oil AK 35 | | |
| EC Erdölchemie GmbH | Isoeicosane | Isoeicosane | 41.9 |
| Wacker | Wacker Silicone | Polydimethylsiloxane | 40.9 |
| | oil AK 20 | | |
| Condea Chemie | Isofol 1212 | | 40.3 |
| | Carbonate | | |
| Gattefossé | Softcutol O | Ethoxydiglycol Oleate | 40.5 |
| Creaderm | Lipodermanol OL | Decyl Olivate | 40.3 |
| Henkel | Cetiol S | Dioctylcyclohexane | 39.0 |
| DEA Mineral oil (Supplier | Pionier 2071 | Mineral Oil | 38.3 |
| Hansen & Rosenthal) | | | |
| Tudapetrol | | | |
| WITCO BV | Hydrobrite 1000 | Paraffinum Liquidum | 37.6 |
| | PO | | |
| Goldschmidt | Tegosoft HP | Isocetyl Palmitate | 36.2 |
| Condea Chemie | Isofol Ester 1693 | | 33.5 |
| Condea Chemie | Isofol Ester 1260 | | 33.0 |
| Dow Corning | Dow Corning Fluid | Cyclopentasiloxane | 32.3 |
| | 245 | | |
| Unichema | Prisorine 2036 | Octyl Isostearate | 31.6 |
| Henkel Cognis | Cetiol CC | Dicaprylyl Carbonate | 31.7 |
| ALZO (ROVI) | Dermol 99 | Trimethylhexyl | 31.1 |
| | | Isononanoate | |
| ALZO (ROVI) | Dermol 89 | 2-Ethylhexyl | 31.0 |
| | | Isononanoate | |
| Unichema | Estol 1540 EHC | Octyl Cocoate | 30.0 |

Of the hydrocarbons, paraffin oil, and further hydrogenated polyolefins, such as hydrogenated polyisobutenes, squalane and squalene, in particular, are to be used advantageously for the purposes of the present invention.

The content of the lipid phase is advantageously chosen to be less than 50% by weight, preferably between 2.5 and 30% by weight, particularly preferably between 5 and 15% by weight, in each case based on the total weight of the preparation. It may also be advantageous, although it is not obligatory, for the lipid phase to comprise up to 40% by weight, based on the total weight of the lipid phase, of polar lipids (having a polarity of ≦20 mN/m) or medium-polarity lipids (having a polarity of from 20 to 30 mN/m).

For the purposes of the present invention, particularly advantageous polar lipids are all native lipids, such as, for example, olive oil, sunflower oil, soybean oil, groundnut oil, rapeseed oil, almond oil, palm oil, coconut oil, castor oil, wheatgerm oil, grapeseed oil, thistle oil, evening primrose oil, macadamia nut oil, corn oil, avocado oil and the like and those listed below.

| | | | Polarity |
|---|---|---|---|
| Manufacturer | Trade name | INCI name | mN/m |
| Condea Chemie | Isofol 14 T | Butyl Decanol (+) Hexyl | 19.8 |
| | | Octanol (+) | |
| | | Hexyl Decanol (+) Butyl | |
| | | Octanol | |
| Lipochemicals | Lipovol MOS- | Tridecyl Stearate(+) | 19.4 |
| INC./USA | 130 | Tridecyl | |
| (Induchem) | | Trimellitate(+) | |
| | | Dipentaerythrityl | |
| | | Hexacaprylate/Hexacaprate | |
| | Castor oil | | 19.2 |
| CONDEA | Isofol Ester | | 19.1 |
| Chemie | 0604 | | |
| Huels | Miglyol 840 | Propylene Glycol | 18.7 |
| CONDEA | | Dicaprylate/Dicaprate | |
| Chemie | | | |
| CONDEA | Isofol 12 | Butyl Octanol | 17.4 |
| Chemie | | | |
| Goldschmidt | Tegosoft SH | Stearyl Heptanoate | 17.8 |
| | Avocado oil | | 14.5 |
| Henkel Cognis | Cetiol B | Dibutyl Adipate | 14.3 |
| ALZO (ROVI) | Dermol 488 | PEG 2 Diethylene | 10.1 |
| | | Hexanoate | |
| Condea Augusta | Cosmacol ELI | C₁₂₋₁₃ Alkyl Lactate | 8.8 |
| S.P.A. | | | |
| ALZO (ROVI) | Dermol 489 | Diethylene Glycol | 8.6 |
| | | Dioctanoate(/ | |
| | | Diisononanoate | |
| Condea Augusta | Cosmacol ETI | Di-C_{12/13} Alkyl Tartrate | 7.1 |
| S.P.A. | | | |
| Henkel Cognis | Emerest 2384 | Propylene Glycol | 6.2 |
| | | Monoisostearate | |
| Henkel Cognis | Myritol 331 | Cocoglycerides | 5.1 |
| Unichema | Prisorine 2041 | Triisostearin | 2.4 |
| | GTIS | | |

Particularly advantageous medium-polar lipids for the purposes of the present invention are those listed below

| | | | Polarity |
|---|---|---|---|
| | | | (Water) |
| Manufacturer | Trade name | INCI name | mN/m |
| Henkel Cognis | Cetiol OE | Dicaprylyl Ether | 30.9 |
| | Dihexyl | Dihexyl Carbonate | 30.9 |
| | carbonate | | |
| Albemarle S.A. | Silkflo 366 NF | Polydecene | 30.1 |
| Stearinerie | DUB VCI 10 | Isodecyl Neopentanoate | 29.9 |
| Dubois Fits | | | |
| ALZO (ROVI) | Dermol IHD | Isohexyl Decanoate | 29.7 |
| ALZO (ROVI) | Dermol 108 | Isodecyl Octanoate | 29.6 |
| | Dihexyl Ether | Dihexyl Ether | 29.2 |
| ALZO (ROVI) | Dermol 109 | Isodecyl 3,5,5 Trimethyl | 29.1 |
| | | Hexanoate | |
| Henkel Cognis | Cetiol SN | Cetearyl Isononanoate | 28.6 |
| Unichema | Isopropyl | Isopropyl Palmitate | 28.8 |
| | palmitate | | |
| Dow Corning | DC Fluid 345 | Cyclomethicone | 28.5 |
| Dow Corning | Dow Corning | Cyclopolydimethylsiloxane | 28.5 |
| | Fluid 244 | | |
| Nikko | Jojoba oil Gold | | 26.2 |
| Chemicals | | | |
| Superior Jojoba | | | |
| Oil Gold | | | |
| Wacker | Wacker | Dimethicone | 26.9 |
| | AK 100 | | |
| ALZO (ROVI) | Dermol 98 | 2-Ethylhexanoic Acid 3,5,5 | 26.2 |
| | | Trimethyl Ester | |
| Dow Corning | Dow Corning | Open | 25.3 |
| | Fluid 246 | | |
| Henkel Cognis | Eutanol G | Octyldodecanol | 24.8 |
| Condea | Isofol 16 | Hexyl Decanol | 24.3 |
| Chemie | | | |
| ALZO (ROVI) | Dermol 139 | Isotridecyl 3,5,5 | 24.5 |
| | | Trimethylhexanoanoate | |
| Henkel Cognis | Cetiol PGL | Hexyldecanol (+) Hexyl | 24.3 |
| | | Decyl Laurate | |
| | Cegesoft C24 | Octyl Palmitate | 23.1 |
| Gattefossé | M.O.D. | Octyldodeceyl Myristate | 22.1 |
| | Macadamia | | 22.1 |
| | Nut Oil | | |
| Bayer AG, | Silicone oil VP | Phenyl Trimethicone | 22.7 |
| Dow Corning | 1120 | | |
| CONDEA | Isocarb 12 | Butyl Octanoic Acid | 22.1 |
| Chemie | | | |
| Henkel Cognis | Isopropyl | Isopropyl Stearate | 21.9 |
| | stearate | | |
| WITCO, | Finsolv TN | C12-15 Alkyl Benzoate | 21.8 |
| Goldschmidt | | | |
| Dr. Straetmans | Dermofeel | Butylene Glycol | 21.5 |
| | BGC | Caprylate/Caprate | |
| Unichema | Miglyol 812 | Caprylic/Capric Triglyceride | 21.3 |
| Huels | | | |
| Trivent (via S. | Trivent OCG | Tricaprylin | 20.2 |
| Black) | | | |
| ALZO (ROVI) | Dermol 866 | PEG ” Diethylhexanoate/ | 20.1 |
| | | Diisononanoate/Ethylhexyl | |
| | | Isononanoate | |

The cosmetic or dermatological preparations according to the invention can also advantageously comprise inorganic particulate hydrophobic or hydrophobicized or oil-absorbing solid-body substances or inorganic gel formers.

Advantageous inorganic particulate hydrophobic or hydrophobicized or oil-absorbing solid-body substances may, for example, be chosen from the group

of inorganic fillers (such as talc, kaolin, zeolites, boron nitride),

of inorganic pigments based on metal oxides or other metal compounds which are sparingly soluble or insoluble in water (in particular oxides of titanium, zinc, iron, manganese, aluminum, cerium),

of inorganic pigments based on silicon oxides (such as, in particular, the grades Aerosil-200, Aerosil 200 V),

of silicate derivatives (such as sodium silicoaluminates or fluoro magnesium silicates (submica grades), calcium aluminum borosilicates). Preference is given here in particular to silica dimethyl silylate (Aerosil® R972).

Aerosils [(fumed silica)=silicon dioxide obtained by thermal decomposition of ethyl silicate)] are highly disperse silicas with an often irregular shape, whose specific surface area is usually very large (200-400 m²/g) and can be controlled depending on the preparation process.

Aerosils to be used particularly advantageously according to the invention are obtainable, for example, under the trade names: Aerosil® 130 (Degussa Huls), Aerosil® 200 (Degussa Huls), Aerosil® 225 (Degussa Huls), Aerosil® 300 (Degussa Huls), Aerosil® 380 (Degussa Huls), B-6C (Suzuki Yushi), CAB-O-SIL Fumed Silica (Cabot), CAB-O-SIL EH-5 (Cabot), CAB-O-SIL HS-5 (Cabot), CAB-O-SIL LM-130 (Cabot), CAB-O-SIL MS-55 (Cabot), CAB-O-SIL M-5 (Cabot), E-6C (Suzuki Yushi), Fossil Flour MBK (MBK), MSS-500 (Kobo), Neosil CT 11 (Crosfield Co.), Ronasphere (Rona/EM Industries), Silica, Anhydrous 31 (Whittaker, Clark & Daniels), Silica, Crystalline 216 (Whittaker, Clark & Daniels), Silotrat-1 (Vevy), Sorbosil AC33 (Crosfield Co.), Sorbosil AC 35 (Crosfield Co.), Sorbosil AC 37 (Crosfield Co.), Sorbosil AC 39 (Crosfield Co.), Sorbosil AC77 (Crosfield Co.), Sorbosil TC 15 (Crosfield Co.), Spherica (Ikeda), Spheriglass (Potters-Ballotini), Spheron L-1500 (Presperse), Spheron N-2000 (Presperse), Spheron P-1500 (Presperse), Wacker HDK H 30 (Wacker-Chemie), Wacker HDK N 20 (Wacker-Chemie), Wacker HDK P 100H (Wacker Silicones), Wacker HDK N 20P (Wacker-Chemie), Wacker HDK N 25P (Wacker-Chemie), Wacker HDK S 13 (Wacker-Chemie) Wacker HDK T 30 (Wacker-Chemie), Wacker HDK V 15 (Wacker-Chemie), Wacker HDK V 15 P (Wacker Chemie), Zelec Sil (DuPont).

It is also advantageous to use those SiO₂ pigments in which the free OH groups on the surface of the particles have been (completely or partially) organically modified. This gives, for example as a result of the addition of dimethylsilyl groups, silica dimethyl silylate (e.g., Aerosil® R972 (Degussa Hüls) Aerosil® R974 (Degussa Hüls), CAB-O-SIL TS-610 (Cabot), CAB-O-SIL TS-720 (Cabot), Wacker HDK H15 (Wacker-Chemie), Wacker HDK H18 (Wacker-Chemie), Wacker HDK H₂O (Wacker-Chemie)). The addition of trimethylsilyl groups gives silica silylate (e.g., Aerosil R 812 (Degussa Huls), CAB-O-SIL TS-530 (Cabot), Sipernat D 17 (Degussa Hüls), Wacker HDK H2000 (Wacker-Chemie)).

Very advantageous inorganic gel formers can, for example, be chosen from the group of modified or unmodified, naturally occurring or synthetic sheet silicates. Although it is entirely favorable to use pure components, the preparations according to the invention may also advantageously comprise mixtures of different modified or unmodified sheet silicates.

Sheet silicates, which are so-called phyllosilicates, are understood for the purposes of this application as meaning silicates and alumosilicates in which the silicate or aluminate units, respectively, are joined together via three Si—O or Al—O bonds and form a waved sheet or layer structure. The fourth Si—O or Al—O valence is saturated by cations. There are relatively weak electrostatic interactions, e.g. hydrogen bridge bonds, between the individual layers. The layer structure is consequently defined largely by strong covalent bonds.

The stoichiometry of the sheet silicates is (Si₂O₃²⁻) for pure silicate structures and (AlₘSi²⁻ₘO₅(^{2+m})⁻) for alumosilicates, where m is a number greater than zero and less than 2.

If no pure silicates are present, but alumosilicates, it should be taken into consideration that each Si⁴⁺ group replaced by Al³⁺ requires a further singly charged cation to neutralize the charge.

The charge balance is preferably balanced by H⁺, alkali metal or alkaline earth metal ions. Aluminum as counterion is also known and advantageous. In contrast to the alumosilicates, these compounds are called aluminum silicates. “Aluminum alumosilicates”, in which aluminum is present both in the silicate network, and also as counterion, are also known and in some cases advantageous for the present invention.

Sheet silicates are well documented in the literature, e.g. in the “Lehrbuch der Anorganischen Chemie” [Textbook of inorganic chemistry], A. F. Hollemann, E. Wiberg and N. Wiberg, 91st-100th edition, Walter de Gruyter—Verlag 1985, passim, and also “Lehrbuch der Anorganischen Chemie” [Textbook of inorganic chemistry], H. Remy, 12th edition, Akademische Verlagsgesellschaft, Leipzig 1965, passim. The layer structure of montmorillonite can be found in Römpps Chemie-Lexikon, Franckh'sche Verlagshandlung W. Keller & Co., Stuttgart, 8th edition, 1985, p. 2668 f.

Examples of sheet silicates are:

| | |
|---|---|
| Montmorillonite | Na_{0.33}((Al_{1.67}Mg_{0.33})(OH)₂(Sᵢ₄O₁₀)) |
| often | Al₂O₃*4SiO₂*H₂O*nH₂O or Al₂[OH)₂/Si₄O₁₀].n H₂O |
| simplified to | |
| Kaolinite | Al₂(OH)₄(Si₂O₅) |
| Ilite | (K₁H₃O)_{y}(Mg₃(OH)₂(Si_{4-y}Al_{y}O₁₀)) |
| and | (K₁H₃O)_{y}(Al₂(OH)₂(Si_{4-y}Al_{y}O₁₀)) |
| | where y = 0.7-0.9 |
| Beidelite | (Ca,Na)_{0.3}(Al₂(OH)₂(Al_{0.5}Si_{3.5}O₁₀)) |
| Nontronite | Na_{0.33}(Fe₂(OH)₂(Al_{0.33}S_{i3.67}O₁₀)) |
| Saponite | (Ca,Na)_{0.33}((Mg,Fe)₃(OH)₂(Al_{0.33}Si_{3.67}O₁₀)) |
| Hectorite | Na_{0.33}(Mg,Li)₃(OH,F)₂(Si₄O₁₀)) |

Montmorillonite represents the main mineral of the naturally occurring bentonites.

Very advantageous inorganic gel formers for the purposes of the present invention are aluminum silicates, such as the montmorillonites (bentonites, hectorites and derivatives thereof, such as quaternium-18 bentonite, quaternium-18 hectorite, stearalkonium bentonite and stearalkonium hectorite) or however magnesium aluminum silicates (Veegum® grades), and sodium magnesium silicates (Laponite® grades).

Montmorillonites represent clay minerals which belong to the dioctahedral smectites, and are masses which swell in water, but do not become plastic. The layer packets in the three-layer structure of the montmorillonites can swell as the result of reversible incorporation of water (in a 2- to 7-fold amount) and other substances such as, for example, alcohols, glycols, pyridine, α-picoline, ammonium compounds, hydroxy-aluminosilicate ions etc.

The chemical formula given above is only approximate; since montmorillonite has a large capacity for ion exchange, Al can be replaced by Mg, Fe²⁺, Fe³⁺, Zn, Pb, Cr, and also Cu and others. The resulting negative charge of the octahedral layers is balanced by cations, in particular Na⁺ (sodium montmorillonite) and Ca²⁺ (calcium montmorillonite is only swellable to a very small degree) in interlayer positions.

Synthetic magnesium silicates and bentonites advantageous for the purposes of the present invention are sold, for example, by Sud-Chemie under the trade name Optigel®.

An aluminum silicate advantageous for the purposes of the present invention is sold, for example, by R.T. Vanderbilt Comp., Inc., under the trade name Veegum®. The various Veegum® grades, which are all advantageous according to the invention, are characterized by the following compositions:

| | (regular grade) | HV | K | HS | S-728 |
|---|---|---|---|---|---|
| SiO₂ | 55.5 | 56.9 | 64.7 | 69.0 | 65.3 |
| MgO | 13.0 | 13.0 | 5.4 | 2.9 | 3.3 |
| Al₂O₃ | 8.9 | 10.3 | 14.8 | 14.7 | 17.0 |
| Fe₂O₃ | 1.0 | 0.8 | 1.5 | 1.8 | 0.7 |
| CaO | 2.0 | 2.0 | 1.1 | 1.3 | 1.3 |
| Na₂O | 2.1 | 2.8 | 2.2 | 2.2 | 3.8 |
| K₂O | 1.3 | 1.3 | 1.9 | 0.4 | 0.2 |
| Ashing loss | 11.1 | 12.6 | 7.6 | 5.5 | 7.5 |

These products swell in water to form viscous gels, which have an alkaline reaction. The organophilization of montmorillonite or bentonites (exchange of the interlayer cations for quaternary alkylammonium ions) produces products (bentones) which are preferably used for dispersion in organic solvents and oils, fats, ointments, paints, coatings and in detergents.

Bentone® is a trade name for various neutral and chemically inert gelling agents which are constructed from long-chain, organic ammonium salts and specific types of montmorillonite.

The following Bentone® grades are sold, for example, from Kronos Titan and are to be used advantageously for the purposes of the present invention:

Bentone® 27, an organically modified montmorillonite, Bentone® 34 (dimethyl-dioctylammonium bentonite), which is prepared in accordance with U.S. Pat. No. 2,531,427 and, because of its lipophilic groups, swells more readily in a lipophilic medium than in water, Bentone® 38, an organically modified montmorillonite, a cream-colored to white powder, Bentone® LT, a purified clay mineral, Bentone® Gel MIO, an organically modified montmorillonite which is supplied as a very fine suspension in mineral oil (SUS-71) (10% bentonite, 86.7% mineral oil and 3.3% wetting agent), Bentone® Gel IPM, an organically modified bentonite which is suspended in isopropyl myristate (10% bentonite, 86.7% isopropyl myristate, 3.3% wetting agent), Bentone® Gel CAO, an organically modified montmorillonite which is taken up in castor oil (10% bentonite, 86.7% castor oil and 3.3% wetting agent), Bentone® Gel Lantrol, an organically modified montmorillonite which, in paste form, is intended for the further processing, in particular for the preparation of cosmetic compositions; 10% bentonite, 64.9 Lantrol (wool wax oil), 22.0 isopropyl myristate, 3.0 wetting agent and 0.1 propyl p-hydroxybenzoate, Bentone® Gel Lan I, a 10% strength Bentone® 27 paste in a mixture of wool wax USP and isopropyl palmitate, Bentone® Gel Lan II, a bentonite paste in pure, liquid wool wax, Bentone® Gel NV, a 15% strength Bentone® 27 paste in dibutyl phthalate, Bentone® Gel OMS, a bentonite paste in Shellsol T., Bentone® Gel OMS 25, a bentonite paste in isoparaffinic hydrocarbons (Idopar® H), Bentone® Gel IPP, a bentonite paste in isopropyl palmitate.

All Bentone grades are to be used advantageously for the purposes of the present invention.

For the purposes of the present invention, preparations may also advantageously comprise one or more hydrocolloids from one or more of the following groups:

organic, natural compounds, such as, for example, agar agar, carrageen, tragacanth, gum arabic, alginates, pectins, polyoses, guar flour, carob bean flour, starch, dextrins, gelatin, casein,

organic, modified natural substances, such as, for example, carboxymethylcellulose and other cellulose ethers, hydroxyethyl cellulose and hydroxypropyl cellulose and microcrystalline cellulose the like,

organic, completely synthetic compounds, such as, for example, polyacrylic and polymethacrylic compounds, vinyl polymers, polycarboxylic acids, polyethers, polyimines, polyamides, polyurethanes.

The cosmetic or dermatological preparations according to the invention can have the customary composition. Particularly advantageous for the purposes of the present invention are skincare preparations: they can be used for cosmetic or dermatological light protection, and also for the treatment of the skin or of the hair and as a makeup product in decorative cosmetics. A further advantageous embodiment of the present invention consists in after-sun products.

Depending on their formulation, cosmetic or topical dermatological compositions for the purposes of the present invention can, for example, be used as skin protection cream, day or night cream etc. It is optionally possible and advantageous to use the compositions according to the invention as bases for pharmaceutical formulations.

Just as emulsions of liquid and solid consistency are used as cosmetic cleansing lotions or cleansing creams, the preparations according to the invention can also represent “cleansing foams”, which can be used, for example, for the removal of makeup or as a mild washing foam—optionally also for blemished skin. Such cleansing foams can also advantageously be applied as so-called “rinse off” preparations, which are rinsed off from the skin following application.

The cosmetic or dermatological preparations according to the invention can also advantageously be in the form of a foam for the care of hair or the scalp, in particular a foam for arranging the hair, a foam which is used while blow-drying the hair, a styling and treatment foam.

For use, the cosmetic and dermatological preparations according to the invention are applied to the skin or the hair in an adequate amount in the manner customary for cosmetics.

The cosmetic or dermatological compositions according to the invention can, for example, be removed from aerosol containers and thereby foamed. Aerosol containers according to the invention are spray devices with a filling of the liquid or slurry-like substances, which are under the pressure of a propellant (pressurized gas or aerosol packagings). Such containers can be provided with valves of very different construction which permit the removal of the contents in the form of a foam.

In addition, the preparations according to the present invention may advantageously be removed from propellant gas-free, mechanically operated pump atomizers (pump dispensers). Particularly advantageous for the purposes of the present invention are pump systems which work without pressurized gas, but with a filter which brings about special swirling.

The cosmetic or dermatological preparations according to the invention can also preferably be removed, for example, from two-chamber aerosol containers and be applied to the skin. Packaging means advantageous according to the invention are containers in which there is one chamber with a filling of the liquid or slurry-like preparations under the pressure of a primary propellant located in a second chamber. Such containers can be equipped with valves of very different construction which permit the removal of the contents of the first chamber in the form of an emulsion or gel in any position—even with the valve pointing downward. An advantageous embodiment are BiCan® aerosol containers in which the product is enclosed in a flexible bag made of metal or plastic within the can.

Post-foaming compositions according to the invention represent unfoamed, i.e. directly after exiting an aerosol container, two- or multiphase systems—generally emulsions. They can be made into foams by gentle rubbing, for example in the hands or during application and rubbing on the skin, but also by stirring or other foaming operations.

Moreover, it has surprisingly been found that in the case of the use of (secondary) propellants, particularly advantageously of propellants soluble in the optionally present oil phase, thus, for example, customary propane/butane mixtures, the preparations according to the invention are not simply sprayed as aerosol droplets, but develop into finely-bubbled, rich foams as soon as such systems containing such (secondary) propellants experience decompression.

In the case of the use of hydrocarbons or mixtures thereof having 4 or 5 carbon atoms, in particular isobutane, n-pentane and isopentane, as (secondary) propellant, it is possible to delay the automatic foaming after exiting from the pressurized packaging.

As a result of the evaporation of the secondary propellant in the applied cosmetic product, heat is additionally withdrawn from the skin and a pleasant cooling effect is achieved. Such post-foaming preparations are therefore likewise regarded as advantageous embodiments of the present invention with independent inventive step.

Suitable pressurized gas containers for the purposes of the present invention are primarily cylindrical vessels made of metal (aluminum, tinplate, contents <1 000 ml), protected or shatter-resistant glass or plastic (contents <220 ml) or shattering glass or plastic (contents <150 ml), in the choice of which compressive strength and breaking strength, corrosion resistance, ease of filling, or ease of sterilizing etc., but also esthetic aspects, handleability, printing properties etc. play a role. The maximum permissible operating pressure of spray cans made of metal at 50° C. is 12 bar and the maximum fill volume at this temperature is about 90% of the total volume. For glass and plastic cans, the values for the operating pressure are lower and dependent on the size of the container and the propellant (whether liquefied, compressed or dissolved gas).

For the purposes of the present invention, cans made of tinplate, aluminum and glass are particularly advantageous. For reasons of corrosion protection, metal cans can be coated on the inside (silver- or gold-coated), for which purpose all standard commercial internal protective coatings are suitable. For the purposes of the present invention, preference is given to polyester, epoxyphenol and polyamide-imide coatings. Film laminates made of polyethylene (PE), polypropylene (PP) or polyethylene terephthalate (PET) on the inside of the cans are also advantageous, in particular for cans made of tinplate.

The pressurized gas containers are usually single-part or two-part, but in most cases three-part cylindrical, conical or differently shaped. If plastics are used as the spray container material, then these should be resistant to chemicals and the sterilization-temperature, gas-tight, impact-resistant and stable to internal pressures in excess of 12 bar. In principle, polyacetals and polyamides are suitable for spray container purposes.

The internal construction of the spray cans and the valve construction are very varied, depending on the intended use and the physical nature of the ingredient—e.g. whether it is in the form of a two-phase or three-phase system—and can be determined by the person skilled in the art by simple exploratory investigations without inventive activity. For suitable variants, reference may be made to the “Aerosol Technologie Handbuch der Aerosol-Verpackung” [Aerosol Technology Handbook of Aerosol Packaging] (Wolfgang Tauscher, Melcher Verlag GmbH Heidelberg/Munich, 1996).

Valves which are advantageous according to the invention can be designed with or without riser tube. The individual components from which valves according to the invention are usually constructed preferably consist of the following materials:

Disk: tinplate: uncoated, gold- or clear-coated, film-laminated (PE, PP or PET) aluminum: uncoated, silver- or gold-coated, different coating variants, Stoner-Mudge design

Seal: natural or synthetic elastomers or thermoplastic (sleeve gaskets, film-lined made of PE or PP) internal and external seals, e.g. made of perbunan, buna, neoprene, butyl, CLB, LDPE, viton, EPDM, chlorobutyl, bromobutyl or diverse compounds

Cone: PA, POM, brass and diverse special materials,

standard bores (e.g.: 0.25 to 0.70 mm or 2×0.45 to 2×1.00 mm),

various shaft diameters

Spring: metal, particularly preferably V2A, stainless steel;

plastic and also elastomer

Casing: standard and impact

VPH bores, RPT bores or slit for overhead applications

materials: e.g. polyacetal, PA, PE, POM and the like

Riser tube: plastic (polymer resin), e.g. PE, PP, PA or polycarbonate

Advantageous spray heads for the purposes of the present invention are, for example, foaming heads for upright use (hold can vertically) or foam heads for overhead application using one or more channels.

Suitable propellants are the customary “classic” readily volatile, liquefied propellant gases, such as, for example, dimethyl ether (DME) and linear or branched-chain hydrocarbons with two to five carbon atoms (such as, in particular, ethane, propane, butane, isobutane and pentane), which can be used on their own or in a mixture with one another.

Compressed air, and also other gases which are under pressure, such as air, oxygen, nitrogen, hydrogen, helium, krypton, xenon, radon, argon, nitrous oxide (N₂O) and carbon dioxide (CO₂) are also advantageously to be used for the purposes of the present invention as propellant gases (either on their own or in any desired mixtures with one another).

The person skilled in the art is naturally aware that there are other propellant gases which are nontoxic per se and which would be suitable in principle for realizing the present invention in the form of aerosol preparations, but which nevertheless should be omitted due to an unacceptable impact on the environment or other accompanying circumstances, in particular halogenated (substituted by fluorine, chlorine, bromine, iodine or astatine) hydrocarbons, such as, for example, fluorocarbons and chlorofluorocarbons (CFCs).

For the purposes of the present invention, said gases can in each case be used individually or in any desired mixtures with one another.

For the purposes of the present invention, the volume fraction of propellant gas is advantageously chosen from the range from 0.1 to 30% by volume, based on the total volume of filler material and propellant gas (corresponding to a volume fraction of from 70 to 99.9% by volume of filler material).

A particularly preferred propellant gas for the purposes of the present invention is carbon dioxide. Foams obtainable from preparations according to the invention which comprise carbon dioxide as one or the active ingredient are particularly advantageous.

Particularly advantageous, finely creamy and rich foams are obtainable when the preparations according to the invention are foamed using linear or branched-chain, halogenated or nonhalogenated hydrocarbons. Very particularly advantageous foams are obtainable by foaming the preparations according to the invention with carbon dioxide, oxygen, compressed air, helium, krypton, xenon, radon, argon or nitrogen (either on their own or in any desired mixtures with one another).

It is particularly advantageous for the purposes of the present invention when the volume fraction of the gas or the gases in the foamed preparation (in the finished foam) is chosen to be from 5 to 80% by volume, based on the total volume of the preparation.

The cosmetic and dermatological preparations according to the invention can comprise cosmetic auxiliaries, as are customarily used in such preparations, e.g. preservatives, preservative assistants, bactericides, perfumes, dyes, pigments which have a coloring effect, moisturizing or humectant substances, fillers which improve the feel on the skin, fats, oils, waxes or other customary constituents of a cosmetic or dermatological formulation, such as alcohols, polyols, polymers, foam stabilizers, electrolytes, organic solvents or silicone derivatives.

Advantageous preservatives for the purposes of the present invention are, for example, formaldehyde donors (such as, for example, DMDM hydantoin), iodopropyl butylcarbamates (e.g. those available under the trade names Koncyl-L, Koncyl-S and Konkaben LMB from Lonza), parabens, phenoxyethanol, ethanol, benzoic acid and the like. According to the invention, the preservative system usually also advantageously comprises preservative assistants, such as, for example, octoxyglycerol, glycine soya etc.

Particularly advantageous preparations are also obtained if antioxidants are used as additives or active ingredients. According to the invention, the preparations advantageously comprise one or more antioxidants. Favorable, but nevertheless optional antioxidants which may be used are all antioxidants customary or suitable for cosmetic or dermatological applications.

For the purposes of the present invention, water-soluble antioxidants, such as, for example, vitamins, e.g. ascorbic acid and derivatives thereof, can be used particularly advantageously.

Preferred antioxidants are also vitamin E and derivatives thereof, and vitamin A and derivatives thereof.

The amount of antioxidants (one or more compounds) in the preparations is preferably 0.001 to 30% by weight, particularly preferably 0.05 to 20% by weight, in particular 0.1 to 10% by weight, based on the total weight of the preparation.

If vitamin E or derivatives thereof are the antioxidant(s), it is advantageous to choose their respective concentrations from the range from 0.001 to 10% by weight, based on the total weight of the formulation.

If vitamin A or vitamin A derivatives, or carotenes or derivatives thereof are the antioxidant(s), it is advantageous to choose their respective concentrations from the range from 0.001 to 10% by weight, based on the total weight of the formulation.

It is particularly advantageous when the cosmetic preparations according to the present invention comprise cosmetic or dermatological active ingredients, preferred active ingredients being antioxidants which can protect the skin against oxidative stress.

Further advantageous active ingredients for the purposes of the present invention are natural active ingredients or derivatives thereof, such as, for example, alpha-lipoic acid, phytoene, D-biotin, coenzyme Q10, alpha-glycosylrutin, carnitine, carnosine, natural or synthetic isoflavonoids, creatine, taurine and β-alanine.

Formulations according to the invention which comprise, for example, known antiwrinkle active ingredients, such as flavone glycosides (in particular α-glycosylrutin), coenzyme Q10, vitamin E or derivatives and the like are particularly advantageously suitable for the prophylaxis and treatment of cosmetic or dermatological changes in the skin, as arise, for example, during skin aging (such as, for example, dryness, roughness and the formation of dryness wrinkles, irritation, reduced refatting (e.g. after washing), visible vascular dilations (telangiectases, couperosis), sagging and formation of lines and wrinkles, local hyperpigmentation, hypopigmentation and abnormal pigmentation (e.g. age spots), increased susceptibility to mechanical stress (e.g. cracking) and the like. They are also advantageously suitable against the appearance of dry or rough skin.

Surprisingly, selected formulations according to the invention can also have an antiwrinkle effect or considerably increase the effect of known antiwrinkle active ingredients. Accordingly, formulations for the purposes of the present invention are particularly advantageously suitable for the prophylaxis and treatment of cosmetic or dermatological skin changes, as arise, for example, during skin aging. They are also advantageously suitable for combating the appearance of dry or rough skin.

In one particular embodiment, the present invention thus relates to products for the care of skin aged in a natural manner, and for the treatment of the secondary damage of photoaging, in particular the phenomena listed above.

The water phase of the preparations according to the invention can advantageously comprise customary auxiliaries, such as, for example, alcohols, in particular those of low carbon number, preferably ethanol or isopropanol, diols or polyols of low carbon number, and ethers thereof, preferably propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes and moisturizers.

Moisturizers is the term used to describe substances or mixtures of substances which, following application or distribution on the surface of the skin, confer on cosmetic or dermatological preparations the property of reducing the moisture loss by the horny layer (also called transepidermal water loss (TEWL)) or have a beneficial effect on the hydration of the horny layer.

Advantageous moisturizers for the purposes of the present invention are, for example, glycerol, lactic acid, pyrrolidone carboxylic acid and urea. In addition, it is particularly advantageous to use polymeric moisturizers from the group of water-soluble and/or water-swellable and/or water-gelable polysaccharides. Particularly advantageous are, for example, hyaluronic acid, chitosan or a fucose-rich polysaccharide which is listed in the Chemical Abstracts under the registry number 178463-23-5 and is available, for example, under the name Fucogel® 1000 from SOLABIA S.A.

The cosmetic and dermatological preparations according to the invention can comprise dyes or color pigments, particularly when they are in the form of decorative cosmetics. The dyes and color pigments can be chosen from the corresponding positive list of the Cosmetics Directive or the EC list of cosmetic colorants. In most cases they are identical to the dyes approved for foodstuffs. Advantageous color pigments are, for example, titanium dioxide, mica, iron oxides (e.g. Fe₂O₃, Fe₃O₄, FeO(OH)) and tin oxide. Advantageous dyes are, for example, carmine, Prussian blue, chromium oxide green, ultramarine blue and manganese violet. It is particularly advantageous to choose the dyes or color pigments from the Rowe Colour Index, 3rd edition, Society of Dyers and Colourists, Bradford, England, 1971.

If the formulations according to the invention are in the form of products which are used on the face, it is favorable to choose one or more substances from the following group: 2,4-dihydroxyazobenzene, 1-(2′-chloro-4′-nitro-1′-phenylazo)-2-hydroxynaphthalene, Ceres red, 2-(sulfo-1-naphthylazo)-1-naphthol-4-sulfonic acid, calcium salt of 2-hydroxy-1,2′-azonaphthalene-1′-sulfonic acid, calcium and barium salts of 1-(2-sulfo-4-methyl-1-phenylazo)-2-naphthylcarboxylic acid, calcium salt of 1-(2-sulfo-1-naphthylazo)-2-hydroxynaphthalene-3-carboxylic acid, aluminum salt of 1-(4-sulfo-1-phenylazo)-2-naphthol-6-sulfonic acid, aluminum salt of 1-(4-sulfo-1-naphthylazo)-2-naphthol-3,6-disulfonic acid, 1-(4-sulfo-1-naphthylazo)-2-naphthol-6,8-disulfonic acid, aluminum salt of 4-(4-sulfo-1-phenylazo)-1-(4-sulfophenyl)-5-hydroxypyrazolone-3-carboxylic acid, aluminum and zirconium salts of 4,5-dibromofluorescein, aluminum and zirconium salts of 2,4,5,7-tetrabromofluorescein, 3′,4′,5′,6′-tetrachloro-2,4,5,7-tetrabromofluorescein and its aluminum salts, aluminum salt of 2,4,5,7-tetraiodofluorescein, aluminum salt of quinophthalonedisulfonic acid, aluminum salt of indigodisulfonic acid, red and black iron oxide (CIN: 77 491 (red) and 77 499 (black)), iron oxide hydrate (CIN: 77 492), manganese ammonium diphosphate and titanium dioxide.

Also advantageous are oil-soluble natural dyes, such as, for example, paprika extracts, β-carotene or cochineal.

Also advantageous for the purposes of the present invention are formulations with a content of pearlescent pigments. Preference is given in particular to the types of pearlescent pigments listed below:

1. Natural pearlescent pigments, such as, for example

“pearl essence” (guanine/hypoxanthin mixed crystals from fish scales) and

“mother of pearl” (ground mussel shells)

2. Monocrystalline pearlescent pigments, such as, for example, bismuth oxychloride (BiOCl)

3. Layer-substrate pigments: e.g. mica/metal oxide

Bases for pearlescent pigments are, for example, pulverulent pigments or castor oil dispersions of bismuth oxychloride or titanium dioxide, and bismuth oxychloride or titanium dioxide on mica. The luster pigment listed under CIN 77163, for example, is particularly advantageous.

Also advantageous are, for example, the following types of pearlescent pigment based on mica/metal oxide:

| Group | Coating/layer thickness | Color |
|---|---|---|
| Silver-white pearlescent | TiO₂: 40-60 nm | silver |
| pigments | | |
| Interference pigments | TiO₂: 60-80 nm | yellow |
| | TiO₂: 80-100 nm | red |
| | TiO₂: 100-140 nm | blue |
| | TiO₂: 120-160 nm | green |
| Color luster pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold shades |
| | TiO₂/Cr₂O₃ | green |
| | TiO₂/Prussian blue | deep blue |
| | TiO₂/carmine | red |

Particular preference is given, for example, to the pearlescent pigments obtainable from Merck under the trade names Timiron, Colorona and Dichrona.

The list of given pearlescent pigments is not of course intended to be limiting. Pearlescent pigments which are advantageous for the purposes of the present invention are obtainable by numerous methods known per se. For example, other substrates apart from mica can be coated with further metal oxides, such as, for example, silica and the like. SiO₂ particles coated with, for example, TiO₂ and Fe₂O₃ (“ronaspheres”), which are marketed by Merck and are particularly suitable for the optical reduction of fine lines are advantageous.

It may, moreover, be advantageous to dispense completely with a substrate such as mica. Particular preference is given to iron pearlescent pigments prepared without the use of mica. Such pigments are obtainable, for example, under the trade name Sicopearl Copper 1000 from BASF.

In addition, also particularly advantageous are effect pigments which are obtainable under the trade name Metasomes Standard/Glitter in various colors (yellow, red, green, blue) from Flora Tech. The glitter particles here are present in mixtures with various auxiliaries and dyes (such as, for example, the dyes with the Color Index (CI) numbers 19140, 77007, 77289, 77491).

The dyes and pigments may be present either individually or in a mixture, and can be mutually coated with one another, different coating thicknesses generally giving rise to different color effects. The total amount of dyes and color-imparting pigments is advantageously chosen from the range from, for example, 0.1% by weight to 30% by weight, preferably from 0.5 to 15% by weight, in particular from 1.0 to 10% by weight, in each case based on the total weight of the preparations.

For the purposes of the present invention, it is also advantageous to provide cosmetic and dermatological preparations whose main purpose is not protection against sunlight, but which nevertheless have a content of UV protectants. Thus, for example, UV-A and/or UV-B filter substances are usually incorporated into day creams or make-up products. UV protectants, like antioxidants, and, if desired, preservatives, also constitute effective protection of the preparations themselves against spoilage. Also favorable are cosmetic and dermatological preparations in the form of a sunscreen.

Accordingly, for the purposes of the present invention, besides comprising one or more UV filter substances according to the invention, the preparations additionally comprise at least one further UV-A and/or UV-B filter substance. The formulations may, although not necessarily, optionally also comprise one or more organic or inorganic pigments as UV filter substances which may be present in the water or oil phase.

The preparations according to the invention can, in addition, also advantageously be in the form of so-called oil-free cosmetic or dermatological emulsions which comprise a water phase and at least one UV filter substance which is liquid at room temperature or one or more silicone derivatives as a further phase. Oil-free formulations for the purposes of the present invention can advantageously also comprise further lipophilic components—such as, for example, lipophilic active ingredients.

Particularly advantageous UV filter substances which are liquid at room temperature for the purposes of the present invention are homomenthyl salicylate (INCI: Homosalate), 2-ethylhexyl 2-cyano-3,3-diphenylacrylate (INCI: Octocrylene), 2-ethylhexyl 2-hydroxybenzoate (2-ethylhexyl salicylate, octyl salicylate, INCI: Octyl Salicylate) and esters of cinnamic acid, preferably 2-ethylhexyl 4-methoxycinnamate (INCI: Octyl Methoxycinnamate) and isopentyl 4-methoxycinnamate (INCI: Isoamyl p-Methoxycinnamate).

Preferred inorganic pigments are metal oxides or other metal compounds which are insoluble or sparingly soluble in water, in particular oxides of titanium (TiO₂), zinc (ZnO), iron (e.g. Fe₂O₃). zirconium (ZrO₂), silicon (SiO₂), manganese (e.g. MnO), aluminum (Al₂O₃), cerium (e.g. Ce₂O₃), mixed oxides of the corresponding metals, and mixtures of such oxides, and also the sulfate of barium (BaSO₄).

The pigments can, for the purposes of the present invention, also advantageously be used in the form of commercially available oily or aqueous redispersions. Dispersion auxiliaries or solubilization promoters may advantageously be added to these predispersions.

According to the invention, the pigments may advantageously by surface-treated (“coated”), the intention being to form or retain, for example, an amphiphilic or hydrophobic character. This surface treatment can consist in providing the pigments with a thin hydrophilic or hydrophobic inorganic or organic layer by processes known per se. The various surface coatings may also comprise water for the purposes of the present invention.

Inorganic surface coatings for the purposes of the present invention may consist of aluminum oxide (Al₂O₃), aluminum hydroxide Al(OH)₃, and aluminum oxide hydrate (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), or iron oxide (Fe₂O₃). These inorganic surface coatings can arise on their own or in combination or in combination with organic coating materials.

Organic surface coatings for the purposes of the present invention may consist of vegetable or animal aluminum stearate, vegetable or animal stearic acid, lauric acid, dimethylpolysiloxane (also: dimethicone), methylpolysiloxane (methicone), simethicone (a mixture of dimethylpolysiloxane with an average chain length of from 200 to 350 dimethylsiloxane units and silica gel) or alginic acid. These organic surface coatings may be present on their own, in combination or in combination with inorganic coating materials.

Zinc oxide particles suitable according to the invention and predispersions of zinc oxide particles are obtainable under the following trade names from the companies listed:

| Trade name | Coating | Manufacturer |
|---|---|---|
| Z - Cote HP1 | 2% dimethicone | BASF |
| Z - Cote | / | BASF |
| ZnO NDM | 5% dimethicone | H&R |

Suitable titanium dioxide particles and predispersions of titanium dioxide particles are obtainable under the following trade names from the companies listed:

| Trade name | Coating | Manufacturer |
|---|---|---|
| MT-100TV | aluminum hydroxide/stearic | Tayca |
| | acid | Corporation |
| MT-100Z | aluminum hydroxide/stearic | Tayca |
| | acid | Corporation |
| Eusolex T-2000 | alumina/simethicone | Merck KgaA |
| Titanium dioxide | octyltrimethylsilane | Degussa |
| T805 | | |
| (Uvinul TiO₂) | | |

An advantageous organic pigment for the purposes of the present invention is 2,2′-methylenebis(6-(2H-benzotriazol-2-yl)-4-(1,1,3,3-tetramethylbutyl)phenol)

[INCI: Bisoctyltriazole], which is available under the trade name Tinosorb® M from CIBA-Chemikalien GmbH.

Advantageous UV-A filter substances for the purposes of the present invention are dibenzoylmethane derivatives, in particular 4-(tert-butyl)₄′-methoxydibenzoylmethane (CAS No. 70356-09-1), which is sold by Givaudan under the name Parsol® 1789 and by Merck under the trade name Eusolex® 9020.

Advantageous further UV filter substances for the purposes of the present invention are sulfonated, water-soluble UV filters, such as, for example,

phenylene-1,4-bis(2-benzimidazyl)-3,3′-5,5′-tetrasulfonic acid and its salts, particularly the corresponding sodium, potassium or triethanolammonium salts, in particular the phenylene-1,4-bis(2-benzimidazyl)-3,3′-5,5′-tetrasulfonic acid bis-sodium salt with the INCI name Bisimidazylate (CAS No.: 180898-37-7), which is available, for example, under the trade name Neo Heliopan AP from Haarmann & Reimer;

salts of 2-phenylbenzimidazole-5-sulfonic acid, such as its sodium, potassium or its triethanolammonium salt, and the sulfonic acid itself with the INCI name Phenylbenzimidazole Sulfonic Acid (CAS No. 27503-81-7), which is available, for example, under the trade name Eusolex 232 from Merck or under Neo Heliopan Hydro from Haarmann & Reimer;

1,4-di(2-oxo-10-sulfo-3-bornylidenemethyl)benzene (also: 3,3′-(1,4-phenylene-dimethylene)bis(7,7-dimethyl-2-oxobicyclo[2.2.1]hept-1-ylmethane sulfonic acid) and salts thereof (particularly the corresponding 10-sulfato compounds, in particular the corresponding sodium, potassium or triethanolammonium salts), which is also referred to as benzene-1,4-di(2-oxo-3-bornylidenemethyl-10-sulfonic acid). Benzene-1,4-di(2-oxo-3-bornylidenemethyl-10-sulfonic acid) has the INCI name Terephthalidene Dicamphor Sulfonic Acid (CAS No.: 90457-82-2) and is available, for example, under the trade name Mexoryl SX from Chimex;

sulfonic acid derivatives of 3-benzylidenecamphor, such as, for example, 4-(2-oxo-3-bornylidenemethyl)benzenesulfonic acid, 2-methyl-5-(2-oxo-3-bornylidene-methyl)sulfonic acid and salts thereof.

Advantageous UV filter substances for the purposes of the present invention are also so-called broadband filters, i.e. filter substances which absorb both UV-A and UV-B radiation.

Advantageous broadband filters or UV-B filter substances are, for example, triazine derivatives, such as, for example,

2,4-bis{[4-(2-ethylhexyloxy)-2-hydroxy]phenyl}-6-(4-methoxyphenyl)-1,3,5-triazine (INCI: Aniso Triazine), which is available under the trade name Tinosorb® S from CIBA Chemikalien GmbH;

dioctylbutylamidotriazone (INCI: Dioctylbutamidotriazone), which is available under the trade name UVASORB HEB from Sigma 3V;

tris(2-ethylhexyl) 4,4′,4″-(1,3,5-triazine-2,4,6-triyltriimino)trisbenzoate, synonym:

2,4,6-tris[anilino(p-carbo-2′-ethyl-1′-hexyloxy)]-1,3,5-triazine (INCI: Octyl Triazone), which is sold by BASF Aktiengesellschaft under the trade name UVINUL® T 150.

An advantageous broadband filter for the purposes of the present invention is 2,2′-methylenebis(6-(2H-benzotriazol-2-yl)-4-(1,1,3,3-tetramethylbutyl)phenol), which is available under the trade name Tinosorb® M from CIBA-Chemikalien GmbH.

A further advantageous broadband filter for the purposes of the present invention is 2-(2H-benzotriazol-2-yl)-4-methyl-6-[2-methyl-3-[1,3,3,3-tetramethyl-1-[(trimethylsilyl)-oxy]disiloxanyl]propyl]phenol (CAS No.: 155633-54-8) with the INCI name Drometrizole Trisiloxane.

The UV filter substances may be oil-soluble or water-soluble.

Advantageous oil-soluble filter substances are, for example:

3-benzylidenecamphor derivatives, preferably 3-(4-methylbenzylidene)camphor, 3-benzylidenecamphor;

4-aminobenzoic acid derivatives, preferably 2-ethylhexyl 4-(dimethyl-amino)benzoate, amyl 4-(dimethylamino)benzoate;

2,4,6-trianilino(p-carbo-2′-ethyl-1′-hexyloxy)-1,3,5-triazine;

esters of benzalmalonic acid, preferably di(2-ethylhexyl) 4-methoxy-benzalmalonate;

esters of cinnamic acid, preferably 2-ethylhexyl 4-methoxycinnamate, isopentyl 4-methoxycinnamate;

derivatives of benzophenone, preferably 2-hydroxy-4-methoxybenzophenone, 2-hydroxy-4-methoxy-4′-methylbenzophenone, 2,2′-dihydroxy-4-methoxy-benzophenone and

UV filters bonded to polymers.

Advantageous water-soluble filter substances are, for example:

sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

A further light protection filter substance to be used advantageously according to the invention is ethylhexyl 2-cyano-3,3-diphenylacrylate (octocrylene), which is obtainable from BASF under the name Uvinul® N 539.

The list of specified UV filters which may be used for the purposes of the present invention is not of course intended to be limiting.

Particularly advantageous preparations for the purposes of the present invention which are characterized by high or very high UV-A protection preferably comprise two or more UV-A and/or broadband filters, in particular dibenzoylmethane derivatives [for example 4-(tert-butyl)-4′-methoxydibenzoylmethane], benzotriazole derivatives [for example 2,2′-methylenebis(6-(2H-benzotriazol-2-yl)-4-(1,1,3,3-tetramethylbutyl)-phenol)], phenylene-1,4-bis(2-benzimidazyl)-3,3′-5,5′-tetrasulfonic acid or its salts, 1,4-di(2-oxo-10-sulfo-3-bornylidenemethyl)benzene or salts thereof and 2,4-bis{[4-(2-ethylhexyloxy)-2-hydroxy]phenyl}-6-(4-methoxyphenyl)-1,3,5-triazine, in each case individually or in any combinations with one another.

The examples below serve to illustrate the present invention without limiting it.

Unless stated otherwise, all amounts, fractions and percentages are based on the weight and the total amount or on the total weight of the preparations.

### EXAMPLE 1

### Foam-Type O/W Cream

| Emulsion I | % by wt. | % by vol. |
|---|---|---|
| Stearic acid | 3.00 | |
| Cetyl alcohol | 8.50 | |
| PEG-20 stearate | 8.50 | |
| Talc | 2.00 | |
| SiO₂ | 2.00 | |
| Polyacrylic acid | 0.20 | |
| Magnesium aluminum silicate | 0.50 | |
| Paraffin oil | 5.00 | |
| Isohexadecane | 2.00 | |
| Dimethicone/vinyldimethicone crosspolymer | 5.00 | |
| PEG-180/Laureth-50/TMMG copolymer | 0.50 | |
| Glycerol | 5.00 | |
| Sodium hydroxide | q.s. | |
| Preservative | q.s. | |
| Perfume | q.s. | |
| Water, demineralized | ad 100.00 | |
| pH adjusted to 6.5-7.5 | | |
| Emulsion I | | 70 |
| Nitrogen | | 30 |

Predispersion of the inorganic gel former and swelling of the hydrocolloid, and of the polymer with stirring in the water phase. Combining of the fatty phase heated to 75° C. with the water phase heated to 70° C. Addition of the particulate hydrophobic, hydrophobicized solid-body substances with stirring. Homogenization by means of a toothed-wheel dispersing machine (rotor-stator principle) at 65° C. 45 min stirring with gassing with nitrogen at 0.7 bar and cooling. Addition of the additives at 30° C. (perfume, active ingredients). Homogenization by means of a toothed-wheel dispersing machine (rotor-stator principle) at 27° C.

### EXAMPLE 2

### Foam-Type O/W Lotion

| Emulsion II | % by wt. | % by vol. |
|---|---|---|
| Stearic acid | 2.00 | |
| Myristyl alcohol | 1.50 | |
| Cetylstearyl alcohol | 0.50 | |
| PEG-100 stearate | 3.00 | |
| Talc | 0.05 | |
| Hydroxyethylcellulose | 0.05 | |
| Magnesium aluminum silicate | 0.20 | |
| Mineral oil | 5.00 | |
| Hydrogenated polyisobutene | 15.0 | |
| Cyclomethicone/polysilicone-11 | 20.00 | |
| PEG-180/Laureth-50/TMMG copolymer | 0.50 | |
| Glycerol | 3.00 | |
| Sodium hydroxide | q.s. | |
| Preservative | q.s. | |
| Perfume | q.s. | |
| Water, demineralized | ad 100.00 | |
| pH adjusted to 5.0-6.5 | | |
| Emulsion II | | 50 |
| Gas (carbon dioxide) | | 50 |

Predispersion of the inorganic gel former and swelling of the hydrocolloid, and of the polymer with stirring in the water phase. Combining of the fatty phase heated to 80° C. with the water phase heated to 72° C. Addition of the particulate hydrophobic, hydrophobicized solid-body substances with stirring. Homogenization by means of a toothed-wheel dispersing machine (rotor-stator principle) at 65° C. 45 min stirring with gassing with carbon dioxide at 1.2 bar and cooling. Addition of the additives at 30° C. (perfume). Homogenization by means of a toothed-wheel dispersing machine (rotor-stator principle) at 30° C.

### EXAMPLE 3

### Foam-Type O/W Lotion

| Emulsion III | % by wt. | % by vol. |
|---|---|---|
| Stearic acid | 5.00 | |
| Cetylstearyl alcohol | 5.50 | |
| PEG-20 stearate | 1.00 | |
| Aluminum starch octenylsuccinate | 3.00 | |
| Al₂O₃ | 0.50 | |
| Talc | 0.50 | |
| Polyurethane | 0.10 | |
| Polyacrylic methacrylate | 0.10 | |
| Magnesium silicate | 0.10 | |
| Cellulose gum | 0.10 | |
| Dimethicone/vinyldimethicone crosspolymer | 5.00 | |
| PEG-180/Octoxynol-40/TMMG copolymer | 0.25 | |
| Cyclomethicone | 5.00 | |
| Isoeicosane | 10.00 | |
| Polydecene | 10.00 | |
| Citric acid | 0.10 | |
| Glycerol | 3.00 | |
| Perfume, preservative, | q.s. | |
| Sodium hydroxide | q.s. | |
| Dyes etc. | q.s. | |
| Water | ad 100.00 | |
| pH adjusted to 6.0-7.5 | | |
| Emulsion III | | 65 |
| Gas (air) | | 35 |

Predispersion of the inorganic gel former and swelling of the hydrocolloids, and of the polymer with stirring in the water phase. Combining of the fatty phase heated to 80° C. with the water phase heated to 75° C. Addition of the particulate hydrophobic, hydrophobicized solid-body substances with stirring. Homogenization by means of a toothed-wheel dispersing machine (rotor-stator principle) at 65° C. 45 min stirring in an open reactor up to 30° C. Addition of the additives at 30° C. (perfume, active ingredients). Homogenization by means of a toothed-wheel dispersing machine (rotor-stator principle) at 25° C.

### EXAMPLE 4

### Foam-Type O/W Emulsion Make-Up

| Emulsion IV | % by wt. | % by vol. |
|---|---|---|
| Palmitic acid | 2.00 | |
| Cetyl alcohol | 2.00 | |
| PEG-100 stearate | 2.00 | |
| Polyacrylic acid | 0.10 | |
| Aluminum starch octenylsuccinate | 0.05 | |
| Manioc starch | 0.05 | |
| Zeolite | 0.75 | |
| Kaolin | 4.50 | |
| Dimethicone/vinyldimethicone crosspolymer | 2.50 | |
| Sodium-magnesium silicate | 0.15 | |
| Polyether-1 | 1.00 | |
| Dimethicone | 10.50 | |
| Paraffin oil | 5.50 | |
| Dicaprylyl ether | 1.00 | |
| Glycerol | 3.00 | |
| Mica | 1.00 | |
| Iron oxides | 1.00 | |
| Titanium dioxide | 4.50 | |
| Vitamin A palmitate | 0.10 | |
| Hectorite | 0.10 | |
| Sodium hydroxide | q.s. | |
| Preservative | q.s. | |
| Perfume | q.s. | |
| Water, demineralized | ad 100.00 | |
| pH adjusted to 6.0-7.5 | | |
| Emulsion IV | | 37 |
| Gas (oxygen) | | 63 |

Predispersion of the inorganic gel formers and swelling of the hydrocolloid, and of the polymer with stirring in the water phase. Combining of the fatty and pigment phase heated to 78° C. with the water phase heated to 75° C. Addition of the particulate hydrophobic, hydrophobicized solid-body substances with stirring. Homogenization by means of a toothed-wheel dispersing machine (rotor-stator principle) at 65° C. 45 min stirring in a Becomix with gassing with oxygen at 1.3 bar with cooling to 30° C. Addition of the aluminum starch octenylsuccinate, the manioc starch, the perfume and the active ingredients at 30° C. Homogenization by means of a toothed-wheel dispersing machine (rotor-stator principle) at 25° C.

### EXAMPLE 5

### Foam-Type O/W Cream

| Emulsion V | % by wt. | % by vol. |
|---|---|---|
| Stearic acid | 4.00 | |
| Cetyl alcohol | 2.00 | |
| PEG-30 stearate | 2.00 | |
| Sorbitan monostearate | 1.50 | |
| Paraffin oil | 5.00 | |
| Cyclomethicone | 1.00 | |
| Vitamin E acetate | 1.00 | |
| Retinyl palmitate | 0.20 | |
| Glycerol | 3.00 | |
| BHT | 0.02 | |
| Na₂H₂EDTA | 0.10 | |
| Dimethicone/Polysilicone-11 | 10.00 | |
| Polyurethane | 0.10 | |
| Carboxymethylcellulose | 0.05 | |
| Polyacrylic acid | 0.10 | |
| Quaternium-18 hectorite | 0.20 | |
| Magnesium aluminum silicates | 0.10 | |
| Silicon dioxide | 0.05 | |
| Talc | 1.00 | |
| Polyether-1 | 0.10 | |
| Perfume, preservative, | q.s. | |
| Dyes | q.s. | |
| Potassium hydroxide | q.s. | |
| Water | ad 100.00 | |
| pH adjusted to 5.0-7.0 | | |
| Emulsion V | | 43 |
| Gas (nitrous oxide) | | 57 |

Predispersion of the inorganic gel formers and swelling of the hydrocolloid, and of the polymer with stirring in the water phase. Combining of the fatty phase heated to 80° C. with the water phase heated to 75° C. Addition of the particulate hydrophobic, hydrophobicized solid-body substances with stirring. Homogenization by means of a toothed-wheel dispersing machine (rotor-stator principle) at 65° C. 45 min stirring in a Becomix with gassing with nitrous oxide at 0.7 bar with cooling to 30° C. Addition of the additives at 30° C. (perfume, active ingredients). Homogenization by means of a toothed-wheel dispersing machine (rotor-stator principle) at 26° C.

### EXAMPLE 6

### Foam-Type O/W Lotion

| Emulsion VI | % by wt. | % by vol. |
|---|---|---|
| Stearic acid | 4.00 | |
| Cetylstearyl alcohol | 1.00 | |
| PEG-100 stearate | 1.00 | |
| Distarch phosphate | 0.50 | |
| Paraffin oil | 6.50 | |
| Cyclomethicones | 2.00 | |
| Dimethicone | 0.50 | |
| Vitamin E acetate | 2.00 | |
| Glycerol | 3.00 | |
| Dimethicone/vinyldimethicone crosspolymer | 1.00 | |
| Carboxymethylcellulose | 0.05 | |
| Polyacrylic acid | 0.10 | |
| Corn starch | 0.10 | |
| Magnesium aluminum silicate | 0.50 | |
| Kaolin | 0.05 | |
| Talc | 0.50 | |
| PEG-180/Laureth-50/TMMG copolymer | 0.50 | |
| Perfume, preservative, dyes etc | q.s. | |
| Sodium hydroxide | q.s. | |
| Water | ad 100.00 | |
| pH adjusted to 6.0-7.5 | | |
| Emulsion VI | | 35 |
| Gas (argon) | | 65 |

Predispersion of the inorganic gel formers and swelling of the hydrocolloid, and of the polymer with stirring in the water phase. Combining of the fatty phase heated to 78° C. with the water phase heated to 75° C. Addition of the particulate hydrophobic, hydrophobicized solid-body substances with stirring. Homogenization by means of a toothed-wheel dispersing machine (rotor-stator principle) at 65° C. 45 min stirring in a Becomix with gassing with argon at 1 bar with cooling to 30° C. Addition of the additives at 30° C. (perfume, active ingredients). Homogenization by means of a toothed-wheel dispersing machine (rotor-stator principle) at 23° C.

### EXAMPLE 7

### Foam-Type Sunscreen Cream

| Emulsion VII | % by wt. | % by vol. |
|---|---|---|
| Stearic acid | 1.00 | |
| Cetylstearyl alcohol | 4.00 | |
| Myristyl alcohol | 1.00 | |
| Boron nitride | 1.00 | |
| Kaolin | 0.50 | |
| Dimethicone/vinyldimethicone crosspolymer | 4.25 | |
| Silica dimethyl silylate | 1.50 | |
| PEG-20 stearate | 1.00 | |
| Acrylate/C₁₀₋₃₀-alkyl crosspolymer | 0.10 | |
| Hectorite | 0.20 | |
| Quaternium-18 hectorite | 0.10 | |
| Caprylic/capric triglycerides | 2.00 | |
| Paraffin oil | 7.50 | |
| Dimethicone | 7.50 | |
| Octyl isostearate | 5.00 | |
| Glycerol | 3.00 | |
| Octyl methoxycinnamate | 4.00 | |
| Butylmethoxydibenzoylmethane | 3.00 | |
| Ethylhexyltriazone | 3.00 | |
| PEG-180/Laureth-50/TMMG copolymer | 1.50 | |
| BHT | 0.02 | |
| Na₂H₂EDTA | 0.10 | |
| Perfume, preservative, | q.s. | |
| dyes etc | q.s. | |
| Potassium hydroxide | q.s. | |
| Water | ad 100.00 | |
| pH adjusted to 5.0-6.0 | | |
| Emulsion VII | | 35 |
| Gas (helium) | | 65 |

Predispersion of the inorganic gel former (hectorites) and swelling of the hydrocolloids and of the polymer with stirring in the water phase. Predispersion of the quaternium-18 hectorite in the hot fatty phase. Combining of the fatty/photoprotective filter phase heated to 78° C. with the water/photoprotective filter phase heated to 75° C. Addition of the particulate hydrophobic, hydrophobicized solid-body substances with stirring. Homogenization by means of a toothed-wheel dispersing machine (rotor-stator principle) at 65° C. 45 min stirring in a Becomix with gassing with helium at 1 bar with cooling to 30° C. Addition of the additives at 30° C. (perfume). Homogenization by means of a toothed-wheel dispersing machine (rotor-stator principle) at 23° C.

That which is claimed:

1. A self-foaming, foam-type, post-foaming or foamable cosmetic or dermatological preparation comprising at least one siloxane elastomer selected from the group consisting of
(i) siloxane elastomers obtainable by reacting vinyl-terminal polymethylsiloxane and methylhydrodimethylsiloxane; and
(ii) siloxane elastomers obtainable by reacting hydroxy-terminal dimethylpolysiloxane and trimethylsiloxy-terminal methylpolysiloxane.

2. The preparation as claimed in claim 1, wherein the total amount of the at least one siloxane elastomer is from 0.01 to 10% by weight, based on the total weight of the preparation.

3. The preparation as claimed in claim 1, further comprising at least one compound selected from the group consisting of oils from hydrocarbons of animal or vegetable origin, synthetic oils, synthetic esters, and synthetic ethers.

4. The preparation as claimed in claim 1, further comprising at least one silicone oil selected from the group consisting of unbranched silicone oils which are pasty or liquid at room temperature and cyclic silicone oils.

5. The preparation as claimed in claim 1, further comprising at least one gas in a total volume fraction of 5 to 80% by volume, based on the total volume of the preparation.

6. The preparation as claimed in claim 5, wherein the at least one gas includes carbon dioxide.

7. The preparation as claimed in claim 1, further comprising at least one moisturizer.

8. The preparation as claimed in claim 1, further comprising an emulsifier system comprising
A. at least one emulsifier A selected from the group consisting of completely neutralized, partially neutralized, and unneutralized, branched and unbranched, saturated and unsaturated fatty acids with a chain length of 10 to 40 carbon atoms,
B. at least one emulsifier B selected from the group consisting of polyethoxylated fatty acid esters with a chain length of 10 to 40 carbon atoms and with a degree of ethoxylation of 5 to 100, and
C. at least one coemulsifier C selected from the group consisting of saturated and unsaturated, branched and unbranched fatty alcohols with a chain length of 10 to 40 carbon atoms.

9. The preparation as claimed in claim 1, further comprising up to 30% by weight—based on the total weight of the preparation—of a lipid phase comprising
(a) one or more lipids selected from the group consisting of silicone oils and silicone waxes, and
(b) one or more lipids selected from the group consisting of nonpolar lipids having a polarity of >30 mN/m,
where the ratio of (a) to (b) is selected from the range of 1:3 to 3:1.

10. The preparation as claimed in claim 1, further comprising at least one UV filtering substance.

11. The preparation as claimed in claim 1, further comprising at least one antioxidant.

12. A self-foaming, foam-type, post-foaming or foamable cosmetic or dermatological preparation comprising a gel comprising
a lipid phase; and
at least one siloxane elastomer selected from the group consisting of
(i) siloxane elastomers obtainable by reacting vinyl-terminal polymethylsiloxane and methylhydrodimethylsiloxane and
(ii) siloxane elastomers obtainable by reacting hydroxy-terminal dimethylpolysiloxane and trimethylsiloxy-terminal methylpolysiloxane.

13. The preparation as claimed in claim 11, wherein the total concentration of the at least one siloxane elastomer is from 0.1 to 60% by weight, based on the total weight of the gel.

14. The preparation as claimed in claim 11, further comprising at least one compound selected from the group consisting of oils from hydrocarbons of animal or vegetable origin, synthetic oils, synthetic esters, and synthetic ethers.

15. The preparation as claimed in claim 11, further comprising at least one silicone oil selected from the group consisting of unbranched silicone oils which are pasty or liquid at room temperature and cyclic silicone oils.

16. The preparation as claimed in claim 11, further comprising at least one gas in a total volume fraction of 5 to 80% by volume, based on the total volume of the preparation.

17. A self-foaming, foam-type, post-foaming or foamable cosmetic or dermatological preparation comprising at least one siloxane elastomer selected from the group consisting of
(a) a siloxane elastomer comprising the units R₂SiO and RSiO_{1.5} wherein each R independently represents a radical selected from the group consisting of hydrogen, C₁₋₂₄-alkyl, aryl, and alkenyl, wherein the weight ratio of the units R₂SiO to RSiO_{1.5} is from 1:1 to 30:1; and
(b) a siloxane elastomer obtainable by the addition reaction of
(i) an organopolysiloxane containing silicon-bonded hydrogen with
(ii) an organopolysiloxane containing unsaturated aliphatic groups,
wherein said siloxane elastomer is insoluble and swellable in silicone oil, and
wherein the amount of hydrogen in organopolysiloxane in (i) above or the amount of unsaturated aliphatic groups in the organopolysiloxane in (ii) above is from 1 to 20 mol % when the organopolysiloxane is noncyclic, or from 1 to 50 mol % when the organopolysiloxane is cyclic.

18. The preparation as claimed in claim 17, wherein the at least one elastomer includes an elastomer from group (a).

19. The preparation as claimed in claim 18, wherein the at least one elastomer from group (a) further comprising one or more units selected from the group consisting of R₃SiO_{0.5} and SiO₂.

20. The preparation as claimed in claim 17, wherein the at least one elastomer includes an elastomer from group (b).

21. The preparation as claimed in claim 17, wherein the total amount of the at least one siloxane elastomer is from 0.01 to 10% by weight, based on the total weight of the preparation.

22. The preparation as claimed in claim 17, further comprising at least one compound selected from the group consisting of oils from hydrocarbons of animal or vegetable origin, synthetic oils, synthetic esters, and synthetic ethers.

23. The preparation as claimed in claim 17, further comprising at least one silicone oil selected from the group consisting of unbranched silicone oils which are pasty or liquid at room temperature and cyclic silicone oils.

24. The preparation as claimed in claim 17, further comprising at least one gas in a total volume fraction of 5 to 80% by volume, based on the total volume of the preparation.

25. A method for increasing the stability of a self-foaming, foam-type, post-foaming or foamable cosmetic or dermatological preparation comprising adding to the preparation at least one siloxane elastomer selected from the group consisting of
(i) siloxane elastomers obtainable by reacting vinyl-terminal polymethylsiloxane and methylhydrodimethylsiloxane and
(ii) siloxane elastomers obtainable by reacting hydroxy-terminal dimethylpolysiloxane and trimethylsiloxy-terminal methylpolysiloxane.

26. The method as claimed in claim 25, wherein the at least one siloxane elastomer is selected from the group consisting of
(a) a siloxane elastomer comprising the units R₂SiO and RSiO_{1.5} wherein each R independently represents a radical selected from the group consisting of hydrogen, C₁₋₂₄-alkyl, aryl, and alkenyl, wherein the weight ratio of the units R₂SiO to RSiO_{1.5} is from 1:1 to 30:1; and
(b) a siloxane elastomer obtainable by the addition reaction of
(i) an organopolysiloxane containing silicon-bonded hydrogen with
(ii) an organopolysiloxane containing unsaturated aliphatic groups,
wherein said siloxane elastomer is insoluble and swellable in silicone oil, and
wherein the amount of hydrogen in organopolysiloxane in (i) above or the amount of unsaturated aliphatic groups in the organopolysiloxane in (ii) above is from 1 to 20 mol % when the organopolysiloxane is noncyclic, or from 1 to 50 mol % when the organopolysiloxane is cyclic.

27. A method for preparing foam, comprising foaming
(a) a preparation comprising at least one siloxane elastomer selected from the group consisting of
(i) siloxane elastomers obtainable by reacting vinyl-terminal polymethylsiloxane and methylhydrodimethylsiloxane and
(ii) siloxane elastomers obtainable by reacting hydroxy-terminal dimethylpolysiloxane and trimethylsiloxy-terminal methylpolysiloxane; and
(b) at least one gas or propellant selected from the group consisting of linear and branched-chain, halogenated or nonhalogenated hydrocarbons, carbon dioxide, oxygen, compressed air, helium, krypton, xenon, radon, argon, nitrogen and dimethyl ether,
by releasing (a) and (b) from a pressurized container.

28. The method for preparing foam as claimed in claim 27, wherein the preparation further comprises at least one compound selected from the group consisting of oils from hydrocarbons of animal or vegetable origin, synthetic oils, synthetic esters, and synthetic ethers.

29. The method for preparing foam as claimed in claim 27, wherein the preparation further comprises at least one silicone oil selected from the group consisting of unbranched silicone oils which are pasty or liquid at room temperature and cyclic silicone oils.
